(19) United States
(12) Patent Application Publication
(10) Pub. No.: US 2006/0167393 A1
(43) Pub. Date: Jul. 27, 2006

(54) Device for the analgesic immobilisation of broken ribs

(75) Inventor: Kalman Bolla, Rheinfall (CH)

Correspondence Address: YOUNG & THOMPSON, 745 SOUTH 23RD STREET, 2ND FLOOR, ARLINGTON, VA, 22202, US

(73) Assignee: CHRISOFIX AG, NEUHAUSEN (CH)

(21) Appl. No.: 10/547,336

(22) PCT Filed: Mar. 1, 2004
(86) PCT No.: PCT/CH04/00109

§ 371 Date: Sep. 1, 2005

(30) Foreign Application Priority Data
Mar. 3, 2003 (CH) 328/03

(51) Int. Cl. A61F 5/00

(52) U.S. Cl. 602/5; 602/19

(57) ABSTRACT

A device (22) for analgesic immobilization in the event of thorax or rib fractures. The immobilization device (22) includes a flat splint element (24) which covers a large area of the region of the break (19), and is provided with an adhesive layer (26) which is located on the side thereof facing the body and which is used to adhere the immobilization device (22) to the body.

### TECHNICAL FIELD

The present invention relates to the field of medical aids. It comprises a device for analgesic immobilisation of fractured ribs (thorax immobilization device) according to the preamble part of claim 1.

Such a device is known from e.g. U.S. Pat. No. 4,312,334.

### BACKGROUND ART

Ribfractures are very painful, especially if more ribs are fractured simultaneously. The fractured ribs loose their mechanical stability, moreover, in specific cases, such as e.g. window-fractures, they are not capable any more of keeping the chest so thrown out that the lung inside could work undisturbed. This can be noticed especially at breathing when the patient experiences pain und this makes him/her to breath flatly (reduced forced vital capacity, FVC), or (in case of multiple fractures) forcing the patient to breath in a paradox way, in which the chest parts paricipating in breathing move in the opposite direction as usual. As in most of the rib fracture cases no intervention is performed, but natural healing occurs, it is desirable to administer some medicine for killing the pain of the patient in order to achieve better breathing.

It has already been known for a long time that for immobilizing fractured ribs, the side with the fracture in the thorax can be fixed by an adhesive plaster, in order to reduce the movement of the fractured rib, however, this is usually not sufficient. There is a suggestion (GB-A-624,425) to use bundle-like, stretchable stripes instead of the plaster, which can be prestreched by means of a releasable stretching device. However, those immobilizing devices ensure a limited movability in the region of the fracture, but, at the same time, they hinder breathing to a large extent, as well.

The earlier mentioned description U.S. Pat. No. 4,312,334 suggests to bind a frame around the patient the front side of the frame consisting of two vertical, arched supporting elements over the chest. The indented part of the thorax being in the fracture area is drawn out by means of a wire fixed on its one end to the chest and on the other end to the regarding supporting element. In this way, the fractured ribs can be kept in a position suitable for healing, easing the breathing resp. reducing pain.

The draw-backs of this arrangement are partly the necessary intervention and the difficulty in positioning the wire, and partly the hindering of the patient in his/her movements by the streched wire and the frame.

### SUMMARY OF THE INVENTION

Based on the above, the task of the present invention is to create an analgesic immobilizing device for use in thorax fractures eliminating the draw-backs of the devices known, the device is simple to produce, easy to apply, quite safe to use and the application of the device results in a reduction of pain and improvement of breathing, without influencing significantly the free movement of the patient.

The task is solved according to features described in claim 1. The essence of the invention lies in a flat splint element covering the fracture area and possibly the fractured rib(s) and the neighbouring, not fractured ribs as well, which splint is provided with an adhesive layer on its side facing the body suitable for adhering the immobilizing device to the body. The splint element can be adhered to the fractured part of the thorax (fracture area) so that preferably the neighbouring, not fractured parts are also covered. The fractured ribs can be thus secured by the splint element being relatively rigid in itself, and at the same time, can be supported also by the uninjured ribs. This stabilization leads to reducing the pain and can facilitate breathing.

In a preferred embodiment of the invention the splint element can be fitted to the outside contour of the thorax particularly without any additional aid or tool, whereas it preferably contains a deformable plastic plate or a plastically deformable metal plate. This plate increases further the efficiency of the splint and makes its application simpler.

The plastically deformable metal plate is made preferably of aluminium, where the plastically deformable metal plate is corrugated in order to improve local deformability with increasing at the same time the rigidity, and the crests of corrugations of the plate are essentially parallel to the ribs to be treated. Such a splint material has already successful applications for different purposes (WO-A1-97/22312).

The wear of such a splint element can be made more comfortable so that the upper and/or lower side of the splint element is provided with a covering, made preferably of some tissue, or of an elastic foam material particularly provided with open pores. In addition, some perforation can also be made in the splint element in order to achieve better permeability of the immobilizing device.

In order to protect the immobilizing device against external effects, such as water or similar substances, it is preferable to use a protecting foil for covering the upper side of the splint element. This protecting foil can be adhered onto the splint element after applying the splint on the body. A protection of the sides can also be achieved in easy way so that the foil over the splint element sticks out on the sides, and forms a continuous rimstrip, whereas the lower side of the protecting foil is also provided with an adhesive layer in the field of the rimstrip.

In order to reduce further the pain caused by rib fractures it is preferred if the immobilizing device is provided additionally also with some local analgesic substance. For this purpose, pain killers may be contained in pads or cushions coupled to the immobilizing device by a releasable bond. Another possibility is that parts of or the total of the adhesive layer contains a pain killer.

### BRIEF DESCRIPTION OF THE FIGURES

The invention will be explained on the basis of figures showing some embodiments.

FIG. 1 illustrates a very simplified perspective view of a first embodiment of the immobilizing device of the invention for putting to rest position the injured ribs,

FIG. 2 shows a top view of the immobilizing device shown in FIG. 1,

FIG. 3 is a top view from the front of an example of rib fracture showing four ribs from among which the second from the top is fractured,

FIG. 4 shows the rib fracture in FIG. 3 in a simplified section along the line IV-IV with the fracture area,

FIG. 5 is a top view from the front of a second embodiment of the invention showing the immobilizing device adhered to the rib fracture shown in FIG. 3,

FIG. 6 illuestrates the effect of the adhered immobilizing device in a view similar to that in FIG. 4,

FIG. 7 shows an enlarged view of a section through the immobilizing device shown in FIGS. 5 and 6.

### DETAILED DESCRIPTION OF THE INVENTION

The device according to the invention is applied to fractured (thorax fractures) or bruized ribs. In these cases the object is to prevent the movement of the injured ribs in the chest, or at least to reduce it to a great extent. It is especially of advantage that in case of a window-fracture (e.g. when more ribs being in a distance from each other are fractured forming thereby a window in the chest), the paradox breathing characteristic in these cases can be influenced in a positive way.

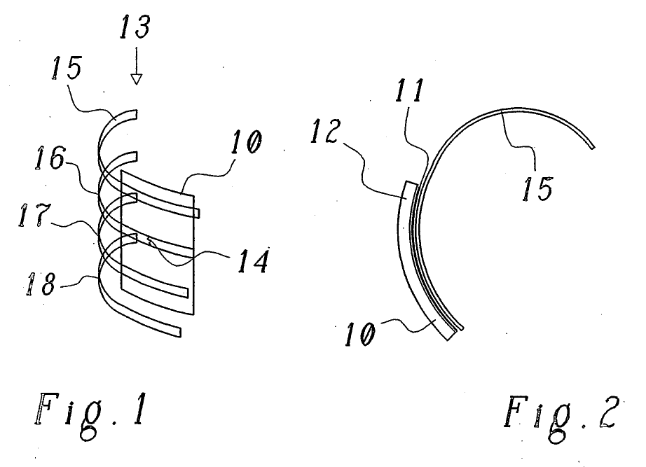

An embodiment of such an immobilizing device and its application are shown in a significantly simplified way in FIGS. 1 and 2. FIG. 1 shows the scheme of four ribs 15-18 from one side of a chest 13, from among which the second rib from the top, rib 16 has a fracture 14. The tissue and skin layers of the body over ribs 15-18 are not shown for simplicity reasons. The intercostal musculature is not shown either. A flat, splint-like immobilizing device 10 fitted to the arching of chest 13 is adhered to the area of chest 13 surrounding fracture 14, on a large part of, or on the total surface. The main component of the immobilizing device 10 consists of a splint element 12 (FIG. 2) in form of a plate made of a suitably rigid but plastically deformable material. Adhering is achieved by applying an appropriate adhesive layer 11 on the inside of splint element 12, similarly to plasters (FIG. 2). The size (lateral dimension) of the immobilizing device 10 is chosen preferably so that the immobilizing device 10 covers not only the injured rib 16, but also the neighbouring ribs 15 and 17 in a sufficient manner.

Through adhering, the immobilizing device 10 is supported by the not fractured part of the injured rib(s) and by the uninjured neighbouring ribs 15 and 17 and keeps the fractured rib 16 in a fixed position relative to the neighbouring ribs 15 and 17. This hinders to a great extent any painful movement of the injured rib 16 at breathing, coughing, laughing or in other similar situations eliminating or at least reducing thereby the pain caused by these movements.

Additionally, some means can also be applied locally to the inside of the immobilizing device 10 for reducing the pain caused by the injured rib 16. Preferably pads or cushions impregnated with some analgesic material having its effect through the skin are used, which are connected to the inside of immobilizing device 10 by a releasable bond, e.g. by adhering or by hook and loop fastener. Another solution may be to impregnate parts of or the total adhesive layer 11 with a suitable pain killer.

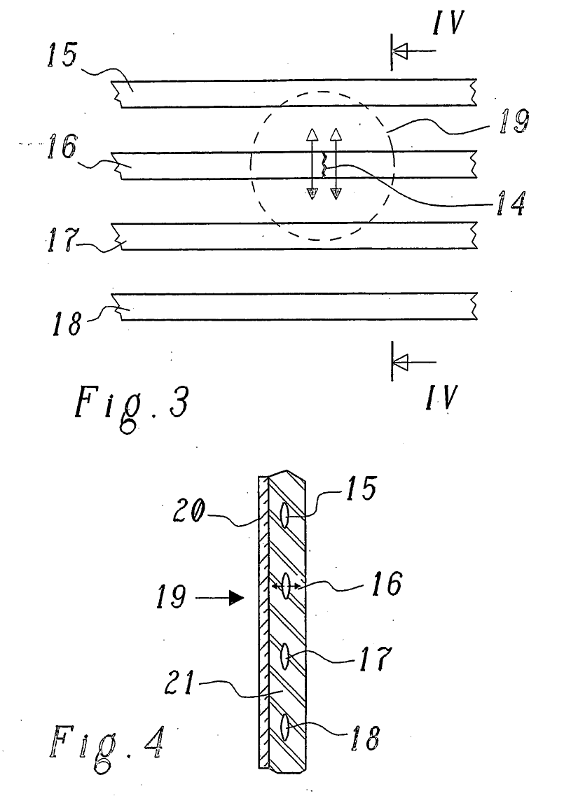

The effect of the immobilizing device 10 according to the present invention may be explained on the basis of FIGS. 3-6. In this case, we also have four parallel ribs 15-18, from among which the second one from the top, rib 16 has a fracture 14 (of course, it is also possible that more fractured ribs are present). Considering the section of the chest along the line IV-IV in FIG. 3, the configuration shown in FIG. 4 is obtained in a simplified form. Ribs 15-18 are embedded into intercostal musculatur 21 serving, among other things, for breathing. This is covered by a multilayer consisting of skin and fat tissues which, in a simplified way, can be denoted as a skin/fat tissue layer 20. In the area of fracture (fracture area 19), the fractured rib 16 looses at least in part its stability, and as a result, a frictional movement (marked in FIGS. 3 and 4 by duble arrows) of the ends of the fracture relatively to each other may occur causing significant pain to the patient at any movement of the chest.

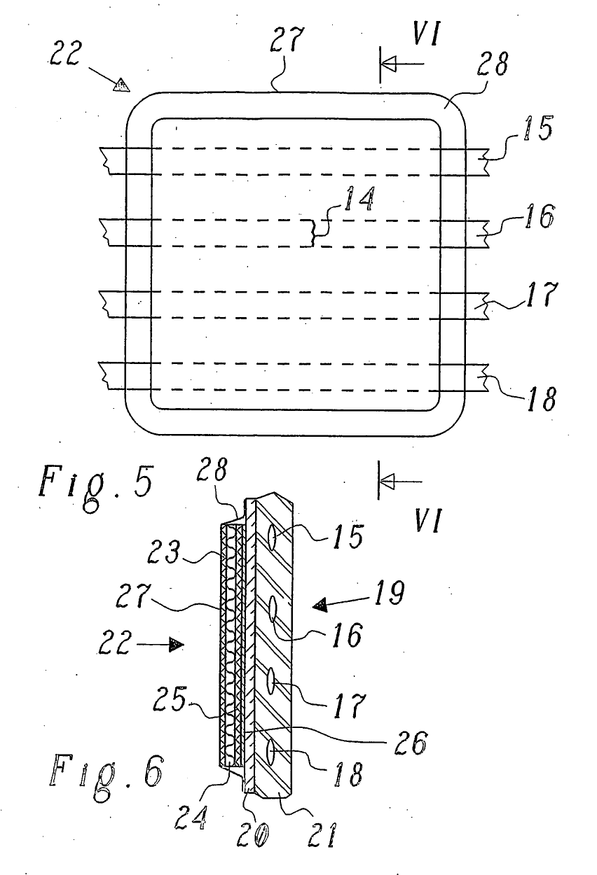

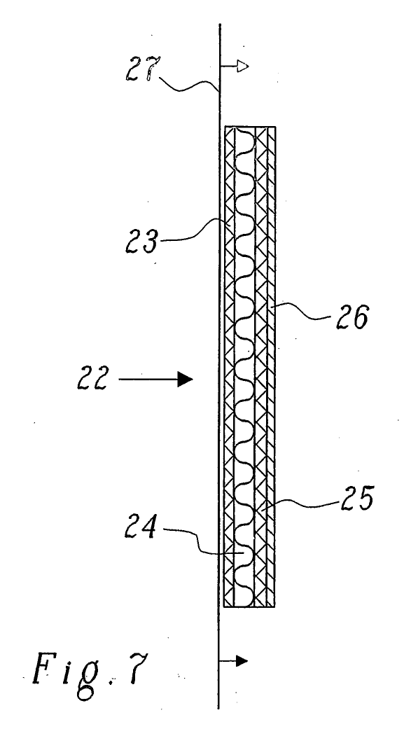

If, according to FIGS. 5 and 6 a flat immobilizing device 22 is adhered to fracture area 19 involving rib 16 and preferably to the not injured ribs 15, 17 and 18 as well, fracture area 19 is stabilized so that rib 16 is immobilized in se and also relative to the other ribs 15, 17 and 18. This leads to a less painful breathing of the patient improving thereby the way of his/her breathing, as well.

Clinical experiments were carried out in 42 patients (33 of them using the immobilizing device, 9 being in the control group) which patients had fractures up to 5 neighbouring ribs, in which experiments the intensity of pain was determined by an analogous scale before the admission of the patients to the study, and 1-2, 24 and 48 hours after that. In comparing with the control group, the intensity of pain in rest (p<0.05), and especially at forced inspiration (p<0.01) was over the whole period significantly less than in the control patients. The reduction of pain owing to the use of immobilizing devices 10 or 22 was measurable already even 1 hour after putting them on, whereas the control patients experienced a measurable reduction of pain only after 2-3 days.

Spirometric measurements were carried out in 18 patients before, and 1-2, 24 and 48 hours after the adhering of the immobilizing device (in several patients in all these periods). Two different sizes of immobilizing devices (12×17 cm and 15×18 cm) were used according to the size of the fracture area. In five further patients (control patients) was the fracture area covered only by operation pads. In these control patients the forced vital capacity (FVC) hindered by the fracture, was further reduced by 174 ml in the average after 1-2 hours, and improved within further 24 or 48 hours only by 4 or 34 ml. To the contrary, in patients treated with the immobilizing device, the FVC continuously and significantly improved (p<0.001), by 153 ml in the average already after 1-2 hours, and by 384 and 474 ml after 24 and 48 hours, after the application of the immobilizing device. Just like FVC, the spirometric parameters FEV1, IVC and PEF improved also by using the immobilizing device.

A preferred embodiment of immobilizing device 22 is shown in FIGS. 5-7. The immobilizing device 22 comprises a flat splint element 24 as central component, in the present case made of a corrugated aluminium plate. The thickness and corrugation of the plate are chosen so that splint element 24 may be fitted easily to the area of the fracture to be treated in the arching of the chest by bare hands without any additional aid, and on the other hand, it is appropriately rigid for its function as support and immobilizing means for the fracture. Splint elements described in WO-A1-97/22312 are also suitable for this purpose (this is why the dates about the material used in that description are taken over in the present application).

In order to fit immobilizing device 22 best to the chest, the crests of the corrugations of splint element 24 are arranged parallel to the ribs. Splint element 24 is provided with a covering 25 on its lower side and covering 23 on its upper side for making its wearing more comfortable. Coverings 23 and 25 are preferably made of an elastic, foamed open-pored or perforated plastic material. Covering 25 at the lower side is provided with an adhesive layer 26 on its outer surface, by means of which the immobilizing device 22 can be adhered to the fracture area. As adhesive materials for the adhesive layer, every adhesive suitable for medical applications can be used. During application, the upper side of the immobilizing device 22, e.g. the outer surface of covering 23 is adhered to a protecting foil 27 which is greater on the sides than the covering, thus forming a protruding rim 28 (FIG. 5). If the protecting foil 27 with its protruding rim 28 is adhered to the skin of the patient, immobilizing device 22 is protected against external effects, thus the patient can e.g. take a shower without any negative consequence. The protecting foil is permeable for air (so called breathing foil) and water-tight. Splint elements 24 in the present invention may be made of other materials than corrugated aluminium plate, such as plastic plates or similar materials being rigid enough and at the same time, sufficiently plastically deformable. Splint element 24 is preferably provided with holes, e.g. in form of a perforation, in order to be permeable and being more comfortable to wear.

### REFERENCE NUMBERS

10, 22 immobilizing device
11 adhesive layer
12 splint element (flat)
13 chest
14 fracture
15-18 ribs
19 fracture area
20 skin/fat tissue layer
21 intercostal musculature
23 upper covering
24 splint element (flat)
25 lower covering
26 adhesive layer
27 protecting foil
28 rim (protecting foil)

1-14. (canceled)

15. Device (10, 22) for analgesic immobilization for use in cases of thorax resp. rib fractures characterized in that the immobilizing device (10, 22) comprises a flat splint element (12, 24) being rigid in itself covering a large part of the fracture area (19) and that the side of immobilizing device (10, 22) facing the body is provided with an appropriate adhesive layer (11, 26) for adhering the immobilizing device (10, 22) to the body.

16. Immobilizing device according to claim 15 characterized in that the splint element (10, 22) can be fitted on the outside contour of the thorax without any additional aid or tool.

17. Immobilizing device according to claim 15 characterized in that the splint element (10, 22) consists of a plastically deformable plastic plate.

18. Immobilizing device according to claim 15 characterized in that the splint element (10, 22) consists of a plastically deformable metal plate.

19. Immobilizing device according to claim 18 characterized in that the plastically deformable metal plate is made of aluminium.

20. Immobilizing device according to claim 19 characterized in that the plastically deformable metal plate is corrugated in order to improve the local deformability enlarging at the same time the rigidity, where the crests of corrugations in the plate run essentially parallel to the ribs to be treated.

21. Immobilizing device according to claim 15 characterized in that the splint element (12, 24) is provided with a covering (23, 25) on its upper and/or lower surface.

22. Immobilizing device according to claim 21 characterized in that the covering (23, 25) at the upper and/or lower surface consists of a tissue or of an elastic, preferably open-pored foam.

23. Immobilizing device according to claim 15 characterized in that it is provided with a protecting foil (27) for protecting covering of the upper side of the splint element (12, 24).

24. Immobilizing device according to claim 23 characterized in that the protecting foil (27) over splint element (12, 24) is developed so that it is larger on the sides forming thereby a surrounding rim (28) as a strip, and that the protecting foil (27) is provided with an adhesive layer on its bottom side.

25. Immobilizing device according to claim 24 characterized in that the immobilizing device (10, 22) comprises also a local analgesic agent.

26. Immobilizing device according to claim 25 characterized in that the analgesic agent is contained in a pad or cushion contacted to the immobilizing device (10, 22) via a releasable bond.

27. Immobilizing device according to claim 25 characterized in that parts of the adhesive layer (11, 26) or the whole adhesive layer (11, 26) is provided with an analgesic agent.
